(19) United States
(12) Patent Application Publication
(10) Pub. No.: US 2025/0032442 A1
(43) Pub. Date: Jan. 30, 2025

(54) METHODS AND COMPOSITIONS FOR TREATING COGNITIVE IMPAIRMENT

(71) Applicant: AXSOME MALTA LTD., Qormi (MT)

(72) Inventors: Scott BOWERSOX, New York, NY (US); Nalina DRONAMRAJU, Philadelphia, PA (US); Wayne MACFADDEN, Philadelphia, PA (US); Eileen LEARY, Palo Alto, CA (US)

(21) Appl. No.: 18/712,835

(22) PCT Filed: Nov. 16, 2022
(86) PCT No.: PCT/US2022/079920

§ 371 Date: May 23, 2024

Related U.S. Application Data
(60) Provisional application No. 63/279,805, filed on Nov. 16, 2021.
(60) Provisional application No. 63/348,684, filed on Jun. 3, 2022.
(60) Provisional application No. 63/378,162, filed on Oct. 3, 2022.

(51) Int. Cl. A61K 31/27; A61K 45/06; A61P 25/28

(52) U.S. Cl. CPC A61K 31/27; A61K 45/06; A61P 25/28

(57) ABSTRACT

The present invention relates to carbamoyl phenylalaninol compounds and methods of using the same to enhance one or more cognitive function and to treat one or more cognitive impairment.

### STATEMENT OF PRIORITY

This application claims the benefit of U.S. Provisional Application Ser. No. 63/279,805, filed Nov. 16, 2021, U.S. Provisional Application Ser. No. 63/348,684, filed Jun. 3, 2022, and U.S. Provisional Application Ser. No. 63/378,162, filed Oct. 3, 2022, the entire contents of each of which are incorporated by reference herein.

### FIELD OF THE INVENTION

The present invention relates to carbamoyl phenylalaninol compounds and methods of using the same to enhance one or more cognitive function and to treat one or more cognitive impairment.

### Background of the Invention

(R)-2-amino-3-phenylpropyl carbamate (APC; solriamfetol; SUNOSI®) is a phenylalanine analog that has been demonstrated to be useful in the treatment of a variety of disorders, including excessive daytime sleepiness, cataplexy, narcolepsy, fatigue, depression, bipolar disorder, fibromyalgia, and others. See, for example, U.S. Pat. Nos. 8,232,315; 8,440,715; 8,552,060; 8,623,913; 8,729,120; 8,741,950; 8,895,609; 8,927,602; 9,226,910; and 9,359,290; and U.S. Publication Nos. 2012/0004300 and 2015/0018414. The structure of the free base of APC is given below as formula I.

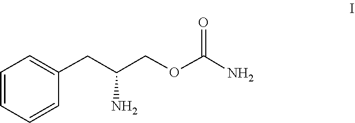 [SMILES: NC(=O)OC[C@H](N)Cc1ccccc1]

Methods for producing APC (which also has other names) and related compounds can be found in U.S. Pat. Nos. 5,955,499; 5,705,640; 6,140,532 and 5,756,817. All of the above patents and applications are hereby incorporated by reference in their entireties for all purposes.

Cognitive impairment is a broad term used to describe some degree of difficulty with cognitive functioning. Impairment can range in intensity and may involve problems with memory, concentration, language, perceptual-motor skills, decision making, and learning. Cognitive impairment is prevalent in our increasingly aging population, with approximately 16% of the population over the age of 70 experiencing some type of mild cognitive impairment. There are currently no FDA-approved treatment for cognitive impairment.

The present invention overcomes shortcomings in the art by providing methods and compositions for enhancing one or more cognitive function and treating one or more cognitive impairment.

### SUMMARY OF THE INVENTION

The present invention relates to the development of methods for enhancing one or more cognitive function and treating one or more cognitive impairment.

Accordingly, one aspect of the present invention relates to a method of enhancing one or more cognitive function, said method comprising administering to a subject in need thereof a therapeutically effective amount of a compound of formula II

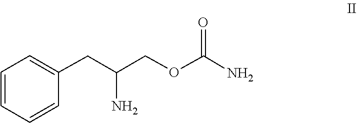 [SMILES: NC(=O)OCC(N)Cc1ccccc1]

or a pharmaceutically acceptable salt thereof.

Another aspect of the invention relates to a method of treating one or more cognitive impairment, said method comprising administering to a subject having said one or more cognitive impairment a therapeutically effective amount of a compound of formula II

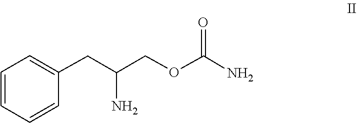 [SMILES: NC(=O)OCC(N)Cc1ccccc1]

or a pharmaceutically acceptable salt thereof.

A further aspect of the invention relates to a method of delaying or slowing an increase in one or more cognitive impairment, said method comprising administering to a subject having or at risk of having said one or more cognitive impairment a therapeutically effective amount of a compound of formula II

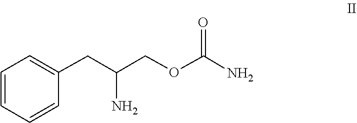 [SMILES: NC(=O)OCC(N)Cc1ccccc1]

or a pharmaceutically acceptable salt thereof, thereby treating one or more cognitive impairment in the subject.

In some embodiments, the compound is the compound of formula I

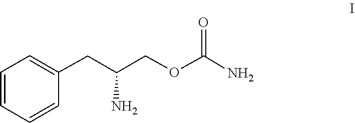 [SMILES: NC(=O)OC[C@H](N)Cc1ccccc1]

or a pharmaceutically acceptable salt thereof. In some embodiments, the compound is the hydrochloride salt of the compound of formula II or I.

These and other aspects of the invention are set forth in more detail in the description of the invention below.

### DETAILED DESCRIPTION OF THE INVENTION

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

All publications, patent applications, patents, and other references mentioned herein are incorporated by reference herein in their entirety for all purposes.

As used herein, “a,” “an,” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

The term “consists essentially of” (and grammatical variants), as applied to the compositions of this invention, means the composition can contain additional components as long as the additional components do not materially alter the composition. The term “materially altered,” as applied to a composition, refers to an increase or decrease in the therapeutic effectiveness of the composition of at least about 20% or more as compared to the effectiveness of a composition consisting of the recited components.

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, and/or change in clinical parameters, disease or illness, etc., as would be well known in the art.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005).

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially.

Concurrent administration can be carried out by mixing the compounds prior to administration, or by administering the compounds in two different formulations, for example, at the same point in time but at different anatomic sites or using different routes of administration.

The present invention relates to a method of enhancing one or more cognitive function, said method comprising administering to a subject in need thereof a therapeutically effective amount of a compound of formula II

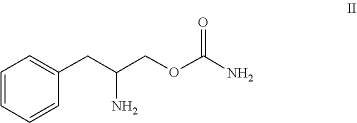 [SMILES: NC(=O)OCC(N)Cc1ccccc1]

or a pharmaceutically acceptable salt thereof.

In some embodiments, the one or more cognitive function is selected from executive function, memory, and learning. In some embodiments, the one or more cognitive function is selected from working memory, cognitive flexibility, and response inhibition.

Another aspect of the invention relates to a method of treating one or more cognitive impairment, said method comprising administering to a subject having said one or more cognitive impairment a therapeutically effective amount of a compound of formula I

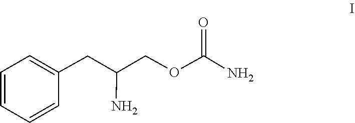 [SMILES: NC(=O)OCC(N)Cc1ccccc1]

or a pharmaceutically acceptable salt thereof.

A further aspect of the invention relates to a method of delaying or slowing an increase in one or more cognitive impairment, said method comprising administering to a subject having or at risk of having said one or more cognitive impairment a therapeutically effective amount of a compound of formula II

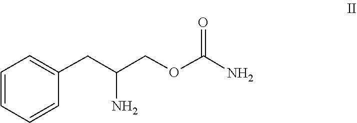 [SMILES: NC(=O)OCC(N)Cc1ccccc1]

or a pharmaceutically acceptable salt thereof, thereby treating one or more cognitive impairment in the subject. The subject may be one that has been diagnosed with one or more cognitive impairment and worsening of the impairments is delayed or slowed relative to a subject not treated with the method of the invention. The subject may be one that is at risk of developing one or more cognitive impairment and onset of the impairments is delayed relative to a subject not treated with the method of the invention.

In some embodiments, the subject is identified as having a neurological disorder associated with cognitive impairment, e.g., identified as having Alzheimer's Disease, schizophrenia, autism, or obsessive-compulsive disorder.

In some embodiments, the cognitive impairment is selected from impaired executive function, impaired learning, and impaired memory. In some embodiments, the cognitive impairment is selected from impaired cognitive flexibility, impaired working memory, and impaired response inhibition.

In some embodiments of the methods of the invention, the compound is the compound of formula I

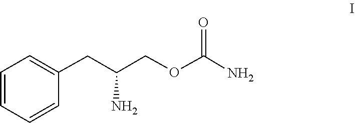 [SMILES: NC(=O)OC[C@H](N)Cc1ccccc1]

or a pharmaceutically acceptable salt thereof. In some embodiments, the compound is the hydrochloride salt of the compound of formula II or I.

The methods of the present invention may be carried out using compounds, formulations and unit dosage forms provided herein. In some embodiments, the formulations and dosage forms can be utilized to achieve immediate release of APC, as well as pharmaceutically acceptable salts, hydrates, isomers, including tautomers, solvates and complexes of APC.

Suitable salts of APC include, without limitation, acetate, adipate, alginate, aspartate, benzoate, butyrate, citrate, fumarate, glycolate, hemisulfate, heptanoate, hexanoate, hydrochloride, hydrobromide, hydroiodide, 2-hydroxyethanesulfonate, lactate, maleate, malonate, methanesulfonate, nicotinate, nitrate, oxalate, palmoate, pectinate, persulfate, hydroxynapthoate, pivalate, propionate, salicylate, succinate, sulfate, tartrate, thiocyanate, tosylate and undecanoate. Other acids, such as oxalic, while not in themselves pharmaceutically acceptable, can be employed in the preparation of salts useful as intermediates in obtaining the compound of the invention and their pharmaceutically acceptable acid addition salts. In certain embodiments, the salt is the hydrochloride salt.

APC compounds include those having quaternization of any basic nitrogen-containing group therein.

The discussion herein is, for simplicity, provided without reference to stereoisomerism or the addition of deuterium atoms. Those skilled in the art will appreciate that APC can contain one or more asymmetric centers and thus occur as racemates and racemic mixtures and single optical isomers. All such isomeric and deuterated forms of these compounds are expressly included in the present invention.

The discussion herein is also provided without reference to polymorphs, hydrates, clathrates, solvates, inclusion compounds, isomers, or other forms of the compound. All such forms of APC are expressly included in the present invention.

Further, the compounds of the invention include prodrugs of the compounds that are converted to the active compound in vivo. For example, the compound can be modified to enhance cellular permeability (e.g., by esterification of polar groups) and then converted by cellular enzymes to produce the active agent. Methods of masking charged or reactive moieties as a pro-drug are known by those skilled in the art (see, e.g., P. Korgsgaard-Larsen and H.

Bundgaard, A Textbook of Drug Design and Development, Reading U.K., Harwood Academic Publishers, 1991).

The term “prodrug” refers to compounds that are rapidly transformed in vivo to yield the parent compound of the above formula, for example, by hydrolysis in blood, see, e.g., T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Exemplary prodrugs include a prodrug that is metabolized in vivo by a subject to an active drug having an activity of the compounds as described herein, wherein the prodrug is an ester of an alcohol or carboxylic acid group, if such a group is present in the compound; an amide of an amine group or carboxylic acid group, if such groups are present in the compound; a urethane of an amine group, if such a group is present in the compound; an acetal or ketal of an alcohol group, if such a group is present in the compound; a N-Mannich base or an imine of an amine group, if such a group is present in the compound; or a Schiff base, oxime, acetal, enol ester, oxazolidine, or thiazolidine of a carbonyl group, if such a group is present in the compound, such as described, for example, in U.S. Pat. Nos. 6,680,324; 6,680,322.

The term “pharmaceutically acceptable prodrug” (and like terms) as used herein refers to those prodrugs of APC which are, within the scope of sound medical judgment, suitable for use in contact with the tissues of humans and/or other animals without undue toxicity, irritation, allergic response and the like, commensurate with a reasonable risk/benefit ratio, and effective for their intended use, as well as the zwitterionic forms, where possible, of the compound of the invention.

APC or a pharmaceutically acceptable salt thereof may be obtained or synthesized by methods known in the art and as described herein. Details of reaction schemes for synthesizing APC have been described in U.S. Pat. Nos. 5,705,640; 5,756,817; 5,955,499; 6,140,532; and 10,829,443, all incorporated herein by reference in their entirety.

Another aspect of the invention relates to a composition, e.g., a dosage form, comprising APC that is suitable for used in the methods of the invention. In some embodiments, the composition is a pharmaceutical composition comprising APC and a pharmaceutically acceptable carrier. In some embodiments, the dosage form is an oral dosage form, e.g., a tablet or a capsule, e.g., an immediate release dosage form.

In some embodiments, the dosage form is an immediate release tablet that releases at least 85%, e.g., at least 85%, 90%, 95%, 96%, 97%, 98%, or 99%, of the APC contained therein within a period of less than 15 minutes after administration of the tablet to a subject, e.g., as described in U.S. Pat. Nos. 10,195,151; 10,512,609; and 11,439,597.

Formulations of APC, including immediate release formulations, may be processed into unit dosage forms suitable for oral administration, such as for example, filled capsules, compressed tablets or caplets, or other dosage form suitable for oral administration using conventional techniques. Immediate release dosage forms prepared as described may be adapted for oral administration, so as to attain and maintain a therapeutic level of the compound over a preselected interval. In certain embodiments, an immediate release dosage form as described herein may comprise a solid oral dosage form of any desired shape and size including round, oval, oblong cylindrical, or polygonal. In one such embodiment, the surfaces of the immediate release dosage form may be flat, round, concave, or convex.

In particular, when the immediate release formulations are prepared as a tablet, the immediate release tablets contain a relatively large percentage and absolute amount of the compound and so are expected to improve patient compliance and convenience, by replacing the need to ingest large amounts of liquids or liquid/solid suspensions. One or more immediate release tablets as described herein can be administered, by oral ingestion, e.g., closely spaced, in order to provide a therapeutically effective dose of the compound to the subject in a relatively short period of time.

Where desired or necessary, the outer surface of an immediate release dosage form may be coated, e.g., with a color coat or with a moisture barrier layer using materials and methods known in the art.

In some embodiments, the composition is an immediate release compressed tablet, the tablet comprising: APC or a pharmaceutically acceptable salt thereof in an amount of about 90-98% by weight of the tablet;
at least one binder in an amount of about 1-5% by weight of the tablet; and
at least one lubricant in an amount of about 0.1-2% by weight of the tablet;
wherein the tablet releases at least 85% of the APC or a pharmaceutically acceptable salt thereof contained therein within a period of less than 15 minutes after administration of the tablet to a subject.

In one embodiment, the tablet comprises: APC or a pharmaceutically acceptable salt thereof in an amount of about 91-95% by weight of the tablet;
at least one binder in an amount of about 2-3% by weight of the tablet;
at least one lubricant in an amount of about 0.1-1% by weight of the tablet; and optionally, a cosmetic film coat in an amount of about 3-4% by weight of the tablet;
wherein the tablet releases at least 85% of the APC or a pharmaceutically acceptable salt thereof contained therein within a period of less than 15 minutes after administration of the tablet to a subject.

In one embodiment, the tablet comprises: APC or a pharmaceutically acceptable salt thereof in an amount of about 93.22% by weight of the tablet;
at least one binder (e.g., hydroxypropylcellulose) in an amount of about 2.87% by weight of the tablet;
at least one lubricant (e.g., magnesium stearate) in an amount of about 0.52% by weight of the tablet; and
optionally, a cosmetic film coat (e.g., Opadry® II yellow) in an amount of about 3-4% by weight of the tablet;
wherein the tablet releases at least 85% of the APC or a pharmaceutically acceptable salt thereof contained therein within a period of less than 15 minutes after administration of the tablet to a subject.

In some embodiments, the composition is an immediate release oral dosage form of APC, the oral dosage form comprising: APC or a pharmaceutically acceptable salt thereof in an amount of about 90-98% by weight of the oral dosage form;
at least one binder in an amount of about 1-5% by weight of the oral dosage form; and
at least one lubricant in an amount of about 0.1-2% by weight of the oral dosage form;
wherein the oral dosage form releases at least 85% of the APC or a pharmaceutically acceptable salt thereof contained therein within a period of less than 15 minutes after administration of the oral dosage form to a subject.

In certain embodiments, the tablet does not comprise a disintegrant. The term “disintegrant,” as used herein, refers to an agent added to a tablet to promote the breakup of the tablet in an aqueous environment. The tablets of the present invention are advantageous in that they dissolve rather than disintegrate. In the present invention the presence of disintegrant in the formulation may actually slow down release of APC.

In certain embodiments, APC or a pharmaceutically acceptable salt thereof is present in an amount of about 90%, 90.5%, 91%, 91.5%, 92%, 92.5%, 93%, 93.5%, 94%, 94.5%, 95%, 95.5%, 96%, 96.5%, 97%, 97.5%, or 98% by weight of the tablet or any value or range therein.

In certain embodiments, APC or a pharmaceutically acceptable salt thereof is present in an amount of about 90% to about 98%, about 92% to about 98%, about 94% to about 98%, about 96% to about 98%, about 90% to about 92%, about 90% to about 94%, about 90% to about 96%, about 92% to about 94%, about 92% to about 96%, or about 94% to about 96%.

In certain embodiments, the at least one binder is present in an amount of about 1%, 1.5%, 2%, 2.5%, 3%, 3.5%, 4%, 4.5%, or 5% by weight of the tablet or any value or range therein. In certain embodiments, the at least one binder is present in an amount of about 1% to about 5%, about 2% to about 5%, about 3% to about 5%, about 4% to about 5%, about 1% to about 2%, about 1% to about 3%, about 1% to about 4%, about 2% to about 3%, about 2% to about 4%, or about 3% to about 4%. The tablet may comprise at least one binder, e.g., 1, 2, 3, 4, 5, or more binders.

In certain embodiments, the at least one binder is selected from at least one of hydroxypropyl cellulose, ethylcellulose, hydroxypropyl methylcellulose, polyvinyl alcohol, hydroxyethyl cellulose, povidone, copovidone, pregelatinized starch, dextrin, gelatin, maltodextrin, starch, zein, acacia, alginic acid, carbomers (cross-linked polyacrylates), polymethacrylates, sodium carboxymethylcellulose, guar gum, hydrogenated vegetable oil (type 1), methylcellulose, magnesium aluminum silicate, and sodium alginate or any combination thereof. In some embodiments, the at least one binder is hydroxypropyl cellulose.

In certain embodiments, the at least one lubricant is present in an amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet or any value or range therein. In certain embodiments, the at least one lubricant is present in an amount of about 0.1% to about 2.0%, about 0.5% to about 2.0%, about 1.0% to about 2.0%, about 1.5% to about 2.0%, about 0.1% to about 0.5%, about 0.1% to about 1.0%, about 0.1% to about 1.5%, about 0.5% to about 1.0%, about 0.5% to about 1.5%, or about 1.0% to about 1.5%. The tablet may comprise at least one lubricant, e.g., 1, 2, 3, 4, 5, or more lubricants. Where the immediate release formulation is provided as a tableted dosage form, still lower lubricant levels may be achieved with use of a “puffer” system during tableting. Such systems are known in the art, commercially available and apply lubricant directly to the punch and die surfaces rather than throughout the formulation.

In certain embodiments, the at least one lubricant is selected from at least one of magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, and zinc stearate or any combination thereof. In some embodiments, the at least one lubricant is magnesium stearate. In other embodiments, magnesium stearate may be used in combination with one or more other lubricants or a surfactant, such as sodium lauryl sulfate. In particular, if needed to overcome potential hydrophobic properties of magnesium stearate, sodium lauryl sulfate may also be included when using magnesium stearate (Remington: the Science and Practice of Pharmacy, 20^{th} edition, Gennaro, Ed., Lippincott Williams & Wilkins (2000)).

In some embodiments, the at least one binder is hydroxypropyl cellulose. In some embodiments, the at least one lubricant is magnesium stearate. In some embodiments, the at least one binder is hydroxypropyl cellulose and the at least one lubricant is magnesium stearate.

In certain embodiments, the tablet is coated. The coating may be, without limitation, a color overcoat.

In some embodiments, the APC or a pharmaceutically acceptable salt thereof is APC hydrochloride.

The tablet may be any shape that is suitable for immediate release and allows the release of at least 85% of the APC or a pharmaceutically acceptable salt thereof contained therein within a period of less than 15 minutes after administration of the tablet to a subject. In some embodiments, the tablet maximizes surface area to volume ratio to promote rapid dissolution. In some embodiments, the tablet is oblong in shape.

The tablet may contain any amount of APC or a pharmaceutically acceptable salt thereof suitable for administration as a unit dosage form. In some embodiments, the tablet contains about 1 mg to about 1000 mg of the drug or any range or value therein, e.g., about 100 mg to about 500 mg, e.g., about 37.5 mg, about 75 mg, about 150 mg, or about 300 mg.

“Immediate release” as used herein, refers to a composition that releases APC or a pharmaceutically acceptable salt, hydrate, isomer, tautomer, solvate or complex thereof substantially completely into the gastrointestinal tract of the user within a period of less than about 15 minutes, usually between about 1 minute and about 15 minutes from ingestion. Such a delivery rate allows the drug to be absorbed by the gastrointestinal tract in a manner that is bioequivalent to an oral solution. Such rapid absorption will typically occur for an immediate release unit dosage form, such as a tablet, caplet or capsule, if the drug included in such dosage form dissolves in the upper portion the gastrointestinal tract.

Release rates can be measured using standard dissolution test methods. For example, the standard conditions may be those described in FDA guidance (e.g., 50 rpm, 37° C., USP 2 paddles, pH 1.2 and pH 6.8 media, 900 ml, 1 test article per vessel).

Immediate release formulations suitable for oral administration may comprise unit dosage forms, such as tablets, caplets or filled capsules, which can deliver a therapeutically effective dose of APC upon ingestion thereof by the patient of one or more of said dosage forms, each of which can provide a dosage of, for example, about 1 to about 1000 mg of APC.

Additionally, the immediate release dosage forms can be shaped or scored to facilitate dose adjustment through tablet splitting.

The formulation and structure of an immediate release dosage form as disclosed herein can be adjusted to provide immediate release performance that suits a particular dosing need.

In particular, the formulation and structure of the dosage forms as described herein can be adjusted to provide any combination of the immediate release performance characteristics described herein. In particular embodiments, for example, an immediate release dosage form as disclosed herein provides rapid onset of action, releasing more than about 85%, such as, for example, more than about 90% or 95%, of the drug contained therein within a period of time selected from less than 15 minutes, less than 12 minutes, less than 10 minutes, and less than 5 minutes after administration.

Moreover, the rate of drug release from an immediate release dosage form as disclosed herein may be adjusted as needed to facilitate a desired dosing regimen or achieve targeted dosing. In one embodiment, the immediate release dosage form may be formulated to deliver as much as 1,000 mg of APC. In particular embodiments, the total amount of drug contained within an immediate release dosage form according to the present description may be between about 50 mg and about 500 mg. For example, in certain such embodiments, the total amount of drug may be selected from about 10, 20, 30, 40, 50, 60, 70, 80, 90, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg or any range or value therein. In certain such embodiments, the total amount of drug may be about 10 mg to about 1000 mg, about 10 mg to about 500 mg, about 10 mg to about 300 mg, about 30 mg to about 1000 mg, about 30 mg to about 500 mg, about 30 mg to about 300 mg, about 100 mg to about 1000 mg, about 10 mg to about 500 mg, about 100 mg to about 300 mg, about 150 mg to about 1000 mg, about 150 mg to about 500 mg, or about 150 mg to about 300 mg.

The immediate release formulations provided herein generally include APC and some level of lubricant to facilitate processing of the formulations into a unit dosage form. In some embodiments, therefore, the formulations described herein include a combination of APC and lubricant, as described herein, and in certain such embodiments, the immediate release formulations are substantially free of other excipients or adjuvants. In other embodiments, the immediate release formulations described herein include a combination of APC, lubricant, and binder, as described herein, and in certain such embodiments, the immediate release formulations are substantially free of other excipients or adjuvants. Though the immediate release formulations described herein may be formulated using a combination of drug and one or more of a lubricant and binder, in certain embodiments, the compositions described herein may include one or more additional excipients selected from, for example, fillers, compression aids, diluents, disintegrants, colorants, flavorants, buffering agents, coatings, glidants, or other suitable excipients.

The immediate release formulations described herein may be manufactured using standard techniques, such as wet granulation, roller compaction, fluid bed granulation, and dry powder blending. Suitable methods for the manufacture of the immediate release formulations and unit dosage forms described herein are provided, for example, in Remington, 20^{th} edition, Chapter 45 (Oral Solid Dosage Forms). It has been found that, even without the aid of binders or non-lubricating excipients, such as compression aids, wet granulation techniques can afford flowable granules with compression characteristics suitable for forming unit dosage forms as described herein. Therefore, in certain embodiments, where a drug content greater than about 85%, 90% or 95% by weight is desired for the immediate release formulation, wet granulation techniques may be used to prepare immediate release formulations as described herein. In such embodiments, as illustrated in the Examples provided herein, conventional organic or aqueous solvents may be used in the wet granulation process. Suitable wet granulation processes can be performed as fluidized bed, high shear, or low shear (wet massing) granulation techniques, as are known in the art.

In addition to one or more of APC, lubricant, and binder, where desired, the immediate release formulations described herein may also include fillers or compression aids selected from at least one of lactose, calcium carbonate, calcium sulfate, compressible sugars, dextrates, dextrin, dextrose, kaolin, magnesium carbonate, magnesium oxide, maltodextrin, mannitol, microcrystalline cellulose, powdered cellulose, and sucrose. Where a filler or compression aid is used, in certain embodiments, it may be included in the immediate release formulation in an amount ranging from about 1%-15% by weight.

Immediate release formulations as described herein may be processed into unit dosage forms suitable for oral administration, such as for example, filled capsules, compressed tablets or caplets, or other dosage form suitable for oral administration using conventional techniques.

Immediate release dosage forms prepared as described may be adapted for oral administration, so as to attain and maintain a therapeutic level of APC over a preselected interval. In certain embodiments, an immediate release dosage form as described herein may comprise a solid oral dosage form of any desired shape and size including round, oval, oblong, cylindrical, or polygonal. In one such embodiment, the surfaces of the immediate release dosage form may be flat, round, concave, or convex. In some embodiments, the shape may be selected to maximize surface area, e.g., to increase the rate of dissolution of the dosage form.

In particular, when the immediate release formulations are prepared as a tablet, the immediate release tablets contain a relatively large percentage and absolute amount of APC and so are expected to improve patient compliance and convenience, by replacing the need to ingest large amounts of liquids or liquid/solid suspensions. One or more immediate release tablets as described herein can be administered, by oral ingestion, e.g., closely spaced, in order to provide a therapeutically effective dose of APC to the subject in a relatively short period of time. For example, dissolution of a 10 mg-1000 mg tablet prepared according to the present description can provide about 80-100% of the APC to the subject in about 10-15 minutes.

Where desired or necessary, the outer surface of an immediate release dosage form as disclosed herein may be coated with a moisture barrier layer using materials and methods known in the art. For example, where the APC delivered by the unit dosage form is highly hygroscopic, providing a moisture barrier layer over the immediate release dosage form as disclosed herein may be desirable. For example, protection of an immediate release dosage form as disclosed herein from water during storage may be provided or enhanced by coating the tablet with a coating of a substantially water soluble or insoluble polymer. Useful water-insoluble or water-resistant coating polymers include ethyl cellulose and polyvinyl acetates.

Further water-insoluble or water-resistant coating polymers include polyacrylates, polymethacrylates or the like. Suitable water-soluble polymers include polyvinyl alcohol and HPMC. Further suitable water-soluble polymers include PVP, HPC, HPEC, PEG, HEC and the like.

Where desired or necessary, the outer surface of an immediate release dosage form as disclosed herein may be coated with a color overcoat or other aesthetic or functional layer using materials and methods known in the art.

The dosage forms disclosed herein can also be provided as a kit comprising, separately packaged, a container comprising a plurality of immediate release tablets, which tablets can be individually packaged, as in foil envelopes or in a blister pack. The tablets can be packaged in many conformations with or without desiccants or other materials to prevent ingress of water.

Instruction materials or means, such as printed labeling, can also be included for their administration, e.g., sequentially over a preselected time period and/or at preselected intervals, to yield the desired levels of APC in vivo for preselected periods of time, to treat a preselected condition.

A daily dose of about 1 to about 2000 mg of APC or a pharmaceutically acceptable salt thereof may be administered to accomplish the therapeutic results disclosed herein. For example, a daily dosage of about 10-1000 mg, e.g., about 20-500 mg, e.g., about 37.5, 75, 150, or 300 mg, in single or divided doses, is administered. In some embodiments, the daily dose may be about 0.01 to about 150 mg/kg body weight, e.g., about 0.2 to about 18 mg/kg body weight.

In one embodiment of the invention, APC is administered to the subject as needed to treat a disorder. The compound can be administered continuously or intermittently. In one embodiment, the compound is administered to the subject more than once a day, e.g., 2, 3, or 4 times per day, or once every 1, 2, 3, 4, 5, 6, or 7 days. In another embodiment, the compound is administered to the subject no more than once a week, e.g., no more than once every two weeks, once a month, once every two months, once every three months, once every four months, once every five months, once every six months, or longer. In a further embodiment, the compound is administered using two or more different schedules, e.g., more frequently initially (for example to build up to a certain level, e.g., once a day or more) and then less frequently (e.g., once a week or less). In other embodiments, the compound can be administered by any discontinuous administration regimen. In one example, the compound can be administered not more than once every three days, every four days, every five days, every six days, every seven days, every eight days, every nine days, or every ten days, or longer. The administration can continue for one, two, three, or four weeks or one, two, or three months, or longer. Optionally, after a period of rest, the compound can be administered under the same or a different schedule. The period of rest can be one, two, three, or four weeks, or longer, according to the pharmacodynamic effects of the compound on the subject. In another embodiment the compound can be administered to build up to a certain level, then maintained at a constant level and then a tailing dosage.

In one aspect of the invention, APC is delivered to a subject concurrently with an additional therapeutic agent. The additional therapeutic agent can be delivered in the same composition as the compound or in a separate composition. The additional therapeutic agent can be delivered to the subject on a different schedule or by a different route as compared to the compound. The additional therapeutic agent can be any agent that provides a benefit to the subject. Further agents include, without limitation, nootropic agents (e.g., cognitive enhancers), including without limitation, stimulants (e.g., caffeine, dimethylamylamine, methylphenidate, amphetamine, nicotine, modafinil, armodafinil), racetams (e.g., piracetam, oxiracetam, phenylpiracetam, aniracetam), tolcapone, levodopa, atomoxetine, pramipexole, guanfacine, clonidine, and fexofenadine. Nootropic agents may also include herbs, such as Bacopa monnieri, Panax ginseng, Ginkgo biloba, and Salvia officinalis, and dietary supplements, such as omega-3 fatty acids, folate, vitamin B6, vitamin B12, vitamin E, and L-theanine. One therapeutic agent that can be administered during the same period is Xyrem®, sold commercially by Jazz Pharmaceuticals, which is used to treat narcolepsy and cataplexy. See U.S. Pat. Nos. 8,952,062 and 9,050,302.

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults and geriatric subjects.

The subject can be a subject “in need of” the methods of the present invention, e.g., in need of the therapeutic effects of the inventive methods. For example, the subject can be a subject that is experiencing cognitive impairment, is suspected of having cognitive impairment, and/or is anticipated to experience cognitive impairment, and the methods and compositions of the invention are used for therapeutic and/or prophylactic treatment.

Having described the present invention, the same will be explained in greater detail in the following examples, which are included herein for illustration purposes only, and which are not intended to be limiting to the invention.

### Example 1

### Cognitive Health Clinical Study

SHARP (Solriamfetol's Effect on Cognitive Health in Apnea Participants During a Randomized Placebo-controlled Study) was a randomized, double-blind, placebo-controlled, crossover, multicenter, trial in which 59 patients with excessive daytime sleepiness (EDS) and obstructive sleep apnea (OSA), who were experiencing cognitive impairment, were all treated with solriamfetol for 2 weeks, and with placebo for 2 weeks, with the treatment periods separated by 1 week of down-titration and washout. Patients were randomized in a 1:1 ratio either to treatment with solriamfetol followed by placebo (sequence 1), or to treatment with placebo followed by solriamfetol (sequence 2). Solriamfetol was administered orally once daily, starting at 75 mg per day for the first three days and 150 mg per day for the remainder of the 2-week treatment period. The primary outcome measure was the Digit Symbol Substitution Test subtest of the Repeatable Battery for the Assessment of Neuropsychological Status (DSST RBANS). The Digit Symbol Substitution subtest is also referred to as “Coding.” The prespecified primary endpoint was the change from baseline in cognitive function as measured by the DSST RBANS after 2 weeks of treatment (average of the 2-, 4-, 6-, and 8-hour post-dose DSST RBANS scores). Secondary endpoints included patient reported measures of cognition including the British Columbia Cognitive Complaints Inventory (BC-CCI) and the Patient Global Impression of Severity (PGI-S) for cognitive symptoms; and the Epworth Sleepiness Scale (ESS) to measure wakefulness. The secondary endpoints were analyzed in a pre-specified testing sequence. All analyses were conducted on an intent-to-treat basis.

Solriamfetol met the primary endpoint in the SHARP study and significantly improved cognitive function, measured using DSST RBANS, as compared to placebo in cognitively impaired patients with EDS associated with OSA. Superiority of solriamfetol as compared to placebo was further demonstrated using patient-reported measures of cognitive function. In the study, solriamfetol replicated previous findings by significantly reducing EDS symptoms as compared to placebo.

On the study's primary endpoint, solriamfetol demonstrated statistically significant improvement in cognitive function compared to placebo as assessed by the change from baseline on the DSST RBANS (6.49 vs. 4.75, p=0.009), with an effect size of 0.36. The DSST RBANS is an objective neuropsychological test that assesses executive function, processing speed and attention. Statistically significant improvement in cognitive function with solriamfetol treatment was also demonstrated using the BC-CCI overall score compared to placebo (p=0.002), with an effect size of 0.43. The BC-CCI is a patient-reported test that assesses domains of memory, concentration, trouble expressing thoughts, word finding, and problem solving.

Solriamfetol significantly improved EDS symptoms compared to placebo, as measured by the Epworth Sleepiness Scale (ESS). The improvement on the ESS with solriamfetol treatment was approximately twice that observed with placebo (p=0.004), with an effect size of 0.50.

The most commonly reported adverse events with solriamfetol treatment (incidence ≥3%) were nausea (6.9%) and anxiety (3.4%). The study completion rate was 96.7% for patients randomized to each treatment sequence (solriamfetol followed by placebo, or placebo followed by solriamfetol).

The foregoing is illustrative of the present invention, and is not to be construed as limiting thereof. The invention is defined by the following claims, with equivalents of the claims to be included therein. All publications, patent applications, patents, patent publications, and any other references cited herein are incorporated by reference in their entireties for the teachings relevant to the sentence and/or paragraph in which the reference is presented.

1-39. (canceled)

40. A method of enhancing one or more cognitive function, said method comprising administering to a subject in need thereof a therapeutically effective amount of a compound of formula II 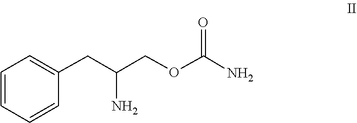 [SMILES: NC(=O)OCC(N)Cc1ccccc1]
or a pharmaceutically acceptable salt thereof, thereby enhancing one or more cognitive function in the subject.

41. The method of claim 40, wherein the compound is the compound of formula |

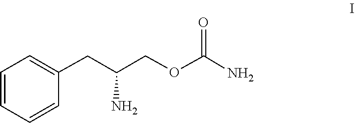 [SMILES: NC(=O)OC[C@H](N)Cc1ccccc1]

or a pharmaceutically acceptable salt thereof.

42. The method of claim 40, wherein the compound is the hydrochloride salt of the compound.

43. The method of claim 40, wherein the compound is administered orally.

44. The method of claim 40, wherein the one or more cognitive function is selected from executive function, memory, and learning.

45. The method of claim 40, wherein the cognitive function is executive function.

46. The method of claim 40, wherein the cognitive function is memory.

47. The method of claim 40, wherein the cognitive function is learning.

48. The method of claim 40, wherein the one or more cognitive function is selected from working memory, cognitive flexibility, and response inhibition.

49. The method of claim 40, wherein the compound is in a pharmaceutical formulation comprising a pharmaceutically acceptable carrier.

50. The method of claim 40, further comprising administering a nootropic agent to the subject in need thereof.

51. A method of treating one or more cognitive impairment, said method comprising administering to a subject having said one or more cognitive impairment a therapeutically effective amount of a compound of formula II 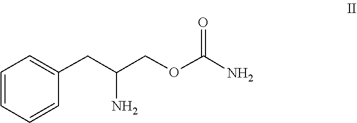 [SMILES: NC(=O)OCC(N)Cc1ccccc1]
or a pharmaceutically acceptable salt thereof, thereby treating one or more cognitive impairment in the subject.

52. The method of claim 51, wherein the compound is the compound of formula I 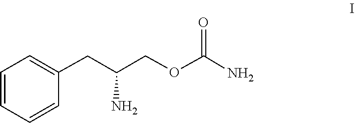 [SMILES: NC(=O)OC[C@H](N)Cc1ccccc1]
or a pharmaceutically acceptable salt thereof.

53. The method of claim 51, wherein the compound is the hydrochloride salt of the compound.

54. The method of claim 51, wherein the subject is identified as having Alzheimer's Disease, schizophrenia, autism, or obsessive-compulsive disorder.

55. The method of claim 51, wherein the cognitive impairment is selected from impaired executive function, impaired learning, and impaired memory.

56. The method of claim 55, wherein the cognitive impairment is impaired executive function.

57. The method of claim 55, wherein the cognitive impairment is impaired learning.

58. The method of claim 55, wherein the cognitive impairment is impaired memory.

59. The method of claim 51, wherein the cognitive impairment is selected from impaired cognitive flexibility, impaired working memory, and impaired response inhibition.

60. The method of claim 59, wherein the cognitive impairment is impaired cognitive flexibility.

61. The method of claim 59, wherein the cognitive impairment is impaired working memory.

62. The method of claim 59, wherein the cognitive impairment is impaired response inhibition.

63. The method of claim 51, wherein the compound is in a pharmaceutical formulation comprising a pharmaceutically acceptable carrier.

64. The method of claim 51, further comprising administering a nootropic agent to the subject having said one or more cognitive impairment.

65. A method of delaying or slowing an increase in one or more cognitive impairment, said method comprising administering to a subject having or at risk of having said one or more cognitive impairment a therapeutically effective amount of a compound of formula ∥ 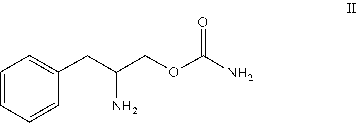 [SMILES: NC(=O)OCC(N)Cc1ccccc1]
or a pharmaceutically acceptable salt thereof, thereby treating one or more cognitive impairment in the subject.

66. The method of claim 65, wherein the compound is the compound of formula I 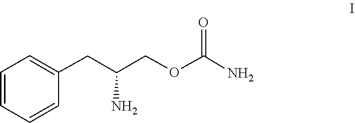 [SMILES: NC(=O)OC[C@H](N)Cc1ccccc1]
or a pharmaceutically acceptable salt thereof.

67. The method of claim 65, wherein the compound is the hydrochloride salt of the compound.

68. The method of claim 65, wherein the subject is identified as having Alzheimer's Disease, schizophrenia, autism, or obsessive-compulsive disorder.

69. The method of claim 65, wherein the cognitive impairment is selected from impaired executive function, impaired learning, and impaired memory.

70. The method of claim 69, wherein the cognitive impairment is impaired executive function.

71. The method of claim 69, wherein the cognitive impairment is impaired learning.

72. The method of claim 69, wherein the cognitive impairment is impaired memory.

73. The method of claim 65, wherein the cognitive impairment is selected from impaired cognitive flexibility, impaired working memory, and impaired response inhibition.

74. The method of claim 73, wherein the cognitive impairment is impaired cognitive flexibility.

75. The method of claim 73, wherein the cognitive impairment is impaired working memory.

76. The method of claim 73, wherein the cognitive impairment is impaired response inhibition.

77. The method of claim 65, wherein the compound is in a pharmaceutical formulation comprising a pharmaceutically acceptable carrier.

78. The method of claim 65, further comprising administering a nootropic agent to the subject having said one or more cognitive impairment.
